(19) United States
(12) Patent Application Publication
(10) Pub. No.: US 2020/0222647 A1
(43) Pub. Date: Jul. 16, 2020

(54) BAROTRAUMA AND VOLUTRAUMA PREVENTION DEVICE

(71) Applicant: THE JOHNS HOPKINS UNIVERSITY, Baltimore, MD (US)

(72) Inventor: Bryan J. Marascalchi, Baltimore, MD (US)

(21) Appl. No.: 16/629,153

(22) PCT Filed: Jul. 6, 2018
(86) PCT No.: PCT/US2018/040985

§ 371 Date: Jan. 7, 2020

Related U.S. Application Data
(60) Provisional application No. 62/529,568, filed on Jul. 7, 2017.

(51) Int. Cl. A61M 16/00; A61B 5/085; A61B 5/087; A61B 5/091

(52) U.S. Cl. CPC A61M 16/0075; A61B 5/085; A61B 5/087; A61M 16/208; A61M 16/0084; A61M 16/024; A61B 5/091

(57) ABSTRACT

An embodiment in accordance with the present invention provides a monitor for use with a bag valve mask (BVM). In some embodiments, the monitor can take the form of an inline electronic spirometer using a bi-directional digital turbine for the BVM with volume, pressure, and respiratory rate alarms and active real time monitoring in order to prevent volutrauma/barotrauma and hypoxia. Alternatively, an out of line electronic spirometer using a variable orifice or fixed orifice flowmeter (pitot tube) and two tubes connected to a portable device can be used. The monitor can come with a BVM or can be a separate device configured for coupling to an existing BVM.

### CROSS REFERENCE TO RELATED APPLICATIONS

This application claims the benefit of U.S. Provisional Patent Application No. 62/529,568 filed Jul. 7, 2017, which is incorporated by reference herein, in its entirety.

### FIELD OF THE INVENTION

The present invention relates generally to medical devices. More particularly, the present invention relates to a device for prevention of barotrauma and volutrauma.

### BACKGROUND OF THE INVENTION

Bag valve masks (BVM) allow for manual ventilation by a caregiver when temporary positive pressure ventilation support is required. The fundamental principal behind this procedure is that a caregiver can estimate the appropriate pressure, volume and frequency of the ventilatory support—simulating what a fully automated ventilator might provide. However, due to a lack of feedback and the very nature of this manual procedure, it is difficult to assess whether the caregiver is over-pressuring and/or over-inflating the patient—the primary cause of additional lung injury. BVMs are un-instrumented and the people using them do not receive feedback on whether they are pushing too hard or too soft, too much or too little. Because of this, lungs can be severely damaged leading to increased hospital stays or even death. The primary complications regarding BVMs are the result of 1) Lung injury from over-stretching (called volutrauma); too much air and 2) Lung injury from over-pressurization (called barotrauma); too much pressure. Complications are on a continuum. Some lead to others, and with worse survivability, prognosis, and even death. The range of injury and spectrum of complications, lead to increased hospitalization, sub-optimal prognosis, and increased expenses.

The following statistics describe the extent of this problem: 1) Patient transport-related adverse events occur 68% of the time (Serious adverse events 8.9%), and 2) When using a BVM all types of caregivers exceed guideline specific pressure limits by more than 5-fold with 88% delivering excessive pressure, 74% delivered excessive volume, and 49% delivering too little respiratory rate.

If the BVM is squeezed too lightly or infrequently, the patient's lungs don't fully expand and will not receive adequate oxygenation. Alternately, if the bag is squeezed too hard, the lungs are over-stretched (called volutrauma from too much volume and/or barotrauma from too much pressure). When users create unfavorable conditions through improper pressure, volume and rate there is an associated cost to the health system per event: Volutrauma and/or barotrauma can lead to adult respiratory distress syndrome (ARDS) in as little as 18 minutes. ARDS is a condition that requires prolonged mechanical ventilator support in the ICU and is associated with poor survival (e.g., 50%), and significantly increased care costs of up to $179,432 per event.

Lung volutrauma from large volumes of air into the lungs can “pop” or collapse the lung (called a pneumothorax), with published reports of BVM causing pneumothoraxes at a cost of up to $9,670.77 per event. In fact, one study presented a case where a large volume of air in the lungs caused a fatal amount of air to enter the pulmonary arteries and heart. Decreased CPR survivability occurs when BVM ventilatory parameters of rate, volume and pressure exceed American Heart Association (AHA) or European Resuscitation Council (ERC) guidelines where the increased volume, pressure, and rate prevents blood from filling the heart.

While the BVM is intended to force-deliver air into the lungs, air may enter the stomach via the esophagus which can inflate if the BVM is under conditions of excessive pressure or volume. This may lead to stomach contents forced into the lungs known as aspiration at a cost of $29,523.27 per event. Aspiration is life-threatening, can require ICU ventilator support and is associated with aspiration pneumonia, ARDS, and chemical pneumonitis.

Therefore, it would be advantageous to provide a device for the prevention of barotrauma and volutrauma.

### SUMMARY OF THE INVENTION

The foregoing needs are met, to a great extent, by the present invention, wherein in one aspect monitor for a bag valve mask comprising an electronic spirometer. The monitor also comprises a display screen.

In accordance with an aspect of the present invention, the electronic spirometer is one selected from a group of an inline spirometer and an out of line spirometer. The display screen can take the form of a touch screen. The electronic spirometer can take the form of a bi-directional digital turbine. The bi-directional digital turbine includes volume, pressure, and respiratory rate alarms. The bi-directional digital turbine is configured to prevent volutrauma/barotrauma and hypoxia. If an out of line spirometer is used, it can include a fixed or variable orifice flowmeter.

In accordance with another aspect of the present invention, a device for delivering positive pressure ventilation includes a mask and a bag. The device can also include an electronic spirometer and a display screen.

### BRIEF DESCRIPTION OF THE DRAWINGS

The accompanying drawings provide visual representations, which will be used to more fully describe the representative embodiments disclosed herein and can be used by those skilled in the art to better understand them and their inherent advantages. In these drawings, like reference numerals identify corresponding elements and:

FIGS. 1 and 2 illustrate perspective views of a device for preventing volutrauma and barotrauma, according to an embodiment of the present invention.

FIGS. 3 and 4 illustrate a display screen according to an embodiment of the present invention.

FIG. 5 illustrates a perspective view between a pressure sensor and a pressure tubing, according to an embodiment of the present invention.

FIGS. 6A and 6B illustrate views of printed circuit boards, according to an embodiment of the present invention.

FIGS. 7A and 7B illustrate perspective views of a device for preventing volutrauma and barotrauma, according to an embodiment of the present invention.

FIG. 8 illustrates a flow diagram of a computer program for operating a device for preventing volutrauma and barotrauma, according to an embodiment of the present invention.

### DETAILED DESCRIPTION

The presently disclosed subject matter now will be described more fully hereinafter with reference to the accompanying Drawings, in which some, but not all embodiments of the inventions are shown. Like numbers refer to like elements throughout. The presently disclosed subject matter may be embodied in many different forms and should not be construed as limited to the embodiments set forth herein; rather, these embodiments are provided so that this disclosure will satisfy applicable legal requirements. Indeed, many modifications and other embodiments of the presently disclosed subject matter set forth herein will come to mind to one skilled in the art to which the presently disclosed subject matter pertains having the benefit of the teachings presented in the foregoing descriptions and the associated Drawings. Therefore, it is to be understood that the presently disclosed subject matter is not to be limited to the specific embodiments disclosed and that modifications and other embodiments are intended to be included within the scope of the appended claims.

An embodiment in accordance with the present invention provides a monitor for use with a bag valve mask (BVM). In some embodiments, the monitor can take the form of an inline electronic spirometer using a bi-directional digital turbine for the BVM with volume, pressure, and respiratory rate alarms and active real time monitoring in order to prevent volutrauma/barotrauma and hypoxia. Alternatively, in some embodiments an out of line electronic spirometer using a fixed orifice flow meter (pitot tube) with two tubes connected to a portable device can be used. A variable orifice flowmeter can also be used. The monitor can come with a BVM or can be a separate device configured for coupling to an existing BVM. Additionally, the device can be used with an endotracheal tube, other airway management device, Mapleson circuit, or any other application known to or conceivable by one of skill in the art.

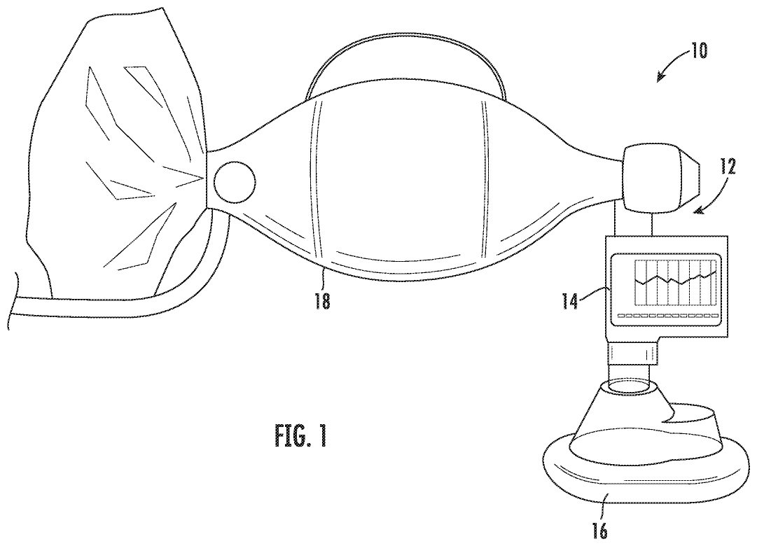

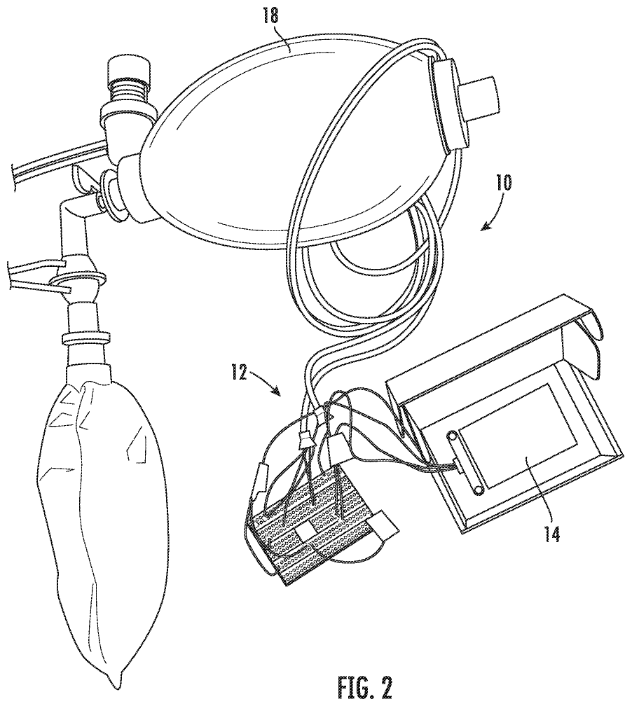

FIGS. 1 and 2 illustrate perspective views of a device for preventing volutrauma and barotrauma, according to an embodiment of the present invention. The device 10 includes a monitoring module 12 and a display screen 14. In a preferred embodiment of the invention, the flow meter is inline with a BVM, The device can be integrated inline with a BVM or configured to couple to an existing BVM. The monitoring module includes a sensor for determining flow and a sensor for determining pressure. In a preferred embodiment, two gauge pressure sensors find the differential pressure and can calculate both pressure and flow. However, multiple configurations of pressure sensors could be envisioned depending on the type of spirometer included on the device. Different configurations could include one or more of the following in combination with other sensors such as ultrasonic flowmeter, light emitting diodes to sense turbine rotations, differential pressure sensors, gauge pressure sensors, or any other pressure sensor type. The monitoring module 12 can take the form of an inline electronic spirometer using a bi-directional digital turbine as well as a pressure sensor. The display screen 14 can include measurements, warnings, and notes for performing the ventilation. In some embodiments, the display screen 14 can include a touch screen that can be used to input information about the patient's size and weight in order to optimize the ventilation for the particular patient. A mask 16 is positionable on the face of the patient and bag 18 is used to deliver air through the mask 16.

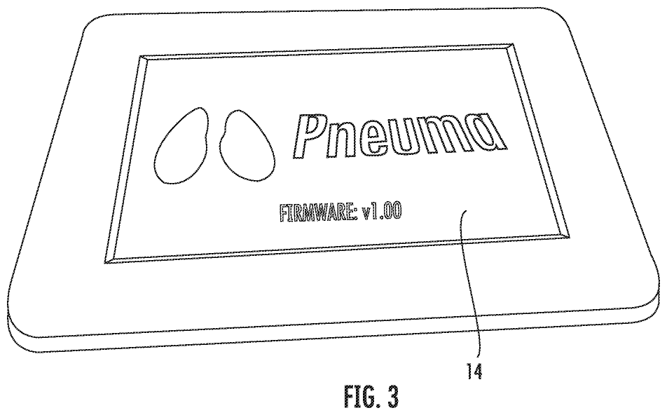

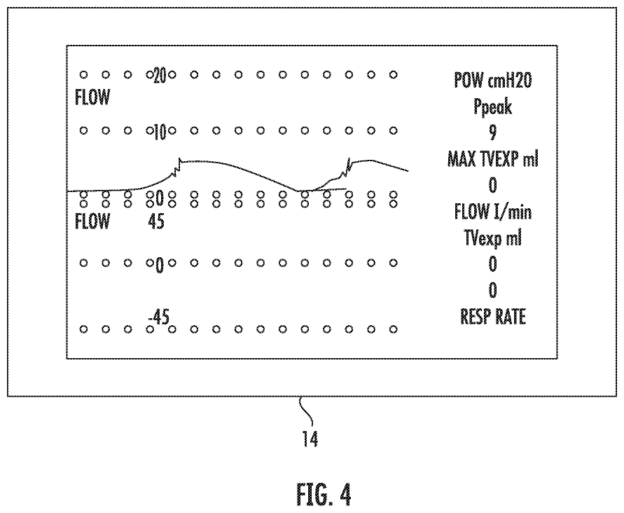

FIGS. 3 and 4 illustrate a display screen according to an embodiment of the present invention. The display screen 14 can include a touchscreen display, in a preferred embodiment. The touchscreen or another interface can be used to enter information about the patient in order to optimize the ventilation for that patient. The display screen 14 also provides relevant information to the healthcare provider, such as, but not limited to airway pressure, flow, tidal volume, respiratory rate, peak pressure, plateau pressure, mean airway pressure, minute ventilation, flow volume loops, and flow and pressure waveforms. The display screen could also be replaced with any other user interface known to or conceivable to one of skill in the art, such as, but not limited to a rotary encoder or buttons.

In some embodiments of the present invention, the BVM barotrauma/volutrauma prevention device could include: a vane anemometer, a variable-orifice flowmeter, a fixed orifice flowmeter (pitot tube), or a hot wire anemometers as the flowmeter. However, these other flow meters have limitations making them unsuitable for continuous respiratory monitoring or the determination of both airway pressure as well as flow. Variable-orifice flowmeters allow for measurement of tidal volumes close to the patient's airway. Several studies have shown that tidal volumes for ventilated patients should be determined with a flow sensor placed at the endotracheal tube due to safety and accuracy. Especially, for small tidal volumes in children, neonates, and adult patients with acute respiratory distress syndrome. Variable-orifice flowmeters allow for a more linear relationship between differential pressure and flow, allowing a larger range of flows to be measured accurately. Variable-orifice flowmeters allows for no or negligible impact of saliva, condensation, blood, or secretions. Whereas, saliva, condensation, blood, and secretions impact the calibration and therefore accuracy of hot wire anemometers, turbine volume transducers and other pneumotachographs. Variable-orifice flowmeters are easily reprocessed, cleaned, or sterilized for reuse, whereas the others aren't or must be disposed.

The bag valve mask barotrauma/volutrauma prevention device uses a variable-orifice flowmeter (a type of pneumotachograph). Pneumotachographs measure flow by finding the pressure drop across the resistance using a differential pressure transducer. Pressure transducers exist in four types differential (finds the difference between two pressures P1 and P2), gauge (finds pressure between P1 and atmospheric pressure, absolute (finds pressure between a perfect vacuum and P1), and vacuum (measures negative gauge pressures). The device of the present invention could use any pneumotachometer. Possible types, include but are not limited to: turbine spirometer/vane anemometer; ultrasonic spirometer; Lilly type pneumotachometer; Fleisch pneumotachometer; variable orifice flowmeter, and fixed orifice flowmeter (pitot tube).

The bag valve mask barotrauma/volutrauma prevention device uses two gauge pressure sensors instead of a differential pressure sensor. This allows for: the simultaneous measure of both airway pressure and flow (tidal volume) close to the patient's airway leading to improved safety and accuracy; and the use of one flow meter to determine both pressure and flow without additional tubing or sensors.

The use of Honeywell ABP digital I²C gauge pressure sensors versus other analog sensors allows for: Calibration over the temperature range of 0° C. to 50° C. [32° F. to 122° F.] so readings do not drift as temperature rises. Readings do not drift as analog circuits increase in temperature from use; Therefore, not requiring re-calibration or a compensation algorithm to correct this effect. Silicone gel coating: allows use in applications where condensation may occur. Other pressure sensors won't last in this environment; High accuracy +/−0.25% without concern for analog noise. Compensated for sensor offset, sensitivity, temperature effects and accuracy errors (which include non-linearity, repeatability and hysteresis); Pressure sensor range should reflect pressures expected from the BVM 0-70 cmH20 in order to have the most accuracy when sensing pressure values. The bag valve mask barotrauma/volutrauma prevention device uses a 0-1 psi [0-70 cmH20] pressure sensor. A different sensor range can also be used depending on the chosen sensors to be included. These particular pressure sensors are included by way of example and are not meant to be considered limiting. Any suitable pressure sensors known to or conceivable to one of skill in the art could also be used.

The main problem is obtaining the two gauge pressures quickly enough that there is no or negligible delay in readings to achieve a differential pressure. The microprocessor code used to request and read data from two gauge sensors and then calculate peak airway pressure and differential pressure (used to find flow and tidal volume) from two gauge sensors simultaneously (and as quickly as possible). Could not find sample code or this method described in literature or by professionals within the field.

The following microprocessor features are required: Two I²C channels (many microprocessors only have one—and I had to switch development platforms for this reason) to allow for either no or negligible delay in readings to achieve a differential pressure. Using one FC channel and slave selection would incorporate unnecessary delay in readings as the microprocessor communicates on one channel or the delay caused by switching from HIGH and LOW on two slave select pins; More than 16 Mhz processor speed: I had to change development platforms from 16 Mhz ATmega to an 180 Mhz ARM Cortex-M4 processor in order to have the speed required to perform all the functions of the device (mainly calculations as quickly as possible). The device can include a real time smoothed z-score algorithm and complex integration of waveforms to calculate tidal volumes, which requires a fast microprocessor. However, one could offload the pressure calculation on to its own microprocessor in order to perform this calculation without interference from other microprocessor functions such as user input or display functions. For this reason, the bag valve mask barotrauma/volutrauma prevention device uses a LCD screen with a built in GPU to handle graphing and drawing functions as well as user I/O (touchscreen processor), to offload these tasks from the microprocessor.

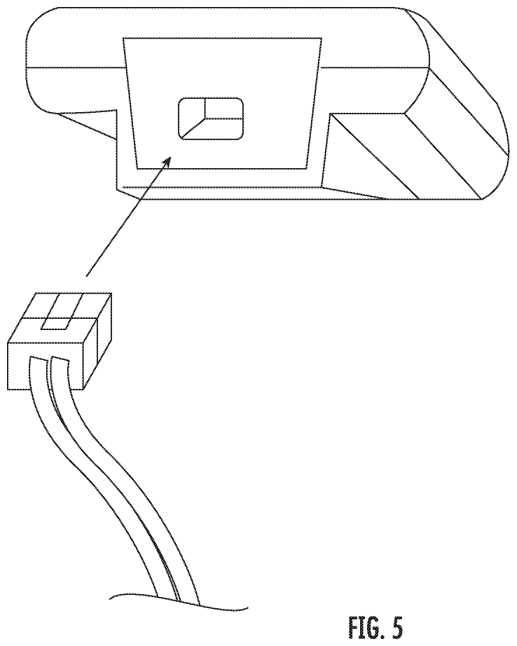

FIG. 5 illustrates a perspective view between a pressure sensor and a pressure tubing, according to an embodiment of the present invention. Leaks at the pressure sensor cause very inaccurate pressure and flow readings making the device unusable, and this connection must be airtight at pressures up to 70 cmH2O. The pressure sensors comes with standard sized axial barbs 0.108″. The pressure tubing has an ID greater than ⅛″ and I had to modifying the pressure sensors by using epoxy to glue metal remote controlled airplane fuel line barbs onto the axial barb of the pressure sensor and use 3/32″ dual ring fuel line hose clamps on this connection to make it airtight. The plastic barb of the pressure sensor with a hose-clamp over the tubing is not airtight. The design illustrated in FIG. 5 shows an airtight connection that can support pressures >70 cmH2O.

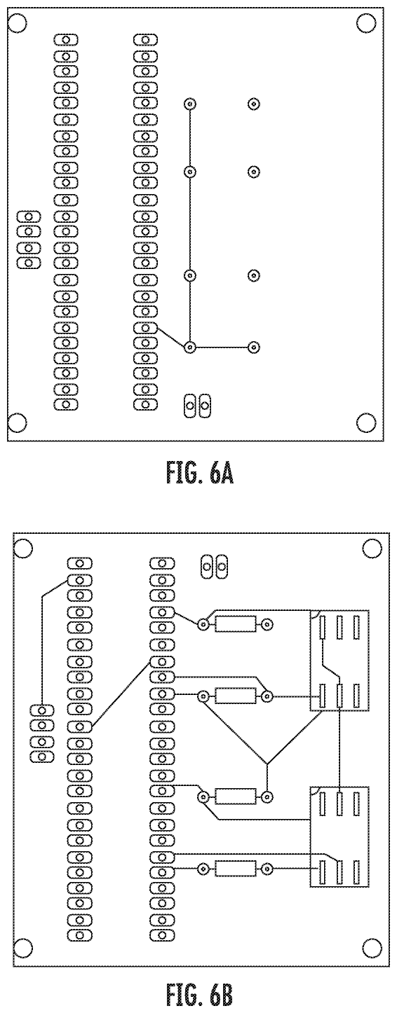

FIGS. 6A and 6B illustrate views of a printed circuit board (PCB), according to an embodiment of the present invention. The device includes circuit boards to control the flowmeter and information going to and from the microprocessors and the display. The PCB is sized to optimized portability of the device. The PCB can also include a power management PCB for charging the battery, determining battery percentage, etc.

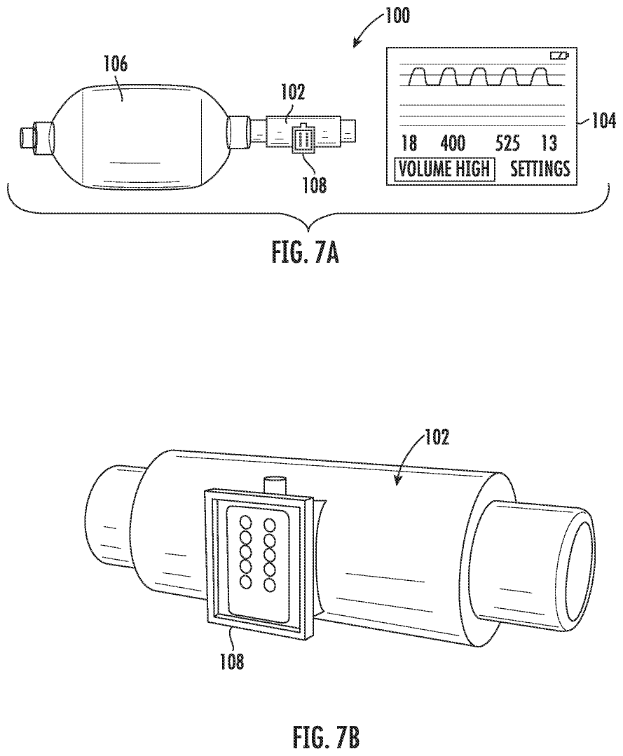

FIGS. 7A and 7B illustrate perspective views of a device for preventing volutrauma and barotrauma, according to an embodiment of the present invention. The device 100 includes a monitoring module 102 and a display screen 104. In a preferred embodiment of the invention, the flow meter is inline with an self-inflating bag 106, The monitoring module 102 can also be integrated inline with a BVM or configured to couple to an existing BVM. Configurations of the monitoring module 102 could include one or more of the following in combination with other sensors such as ultrasonic flowmeter, light emitting diodes to sense turbine rotations, differential pressure sensors, gauge pressure sensors, or any other pressure sensor type. The monitoring module 102 can take the form of an inline electronic spirometer using a bi-directional digital turbine as well as a pressure sensor. The display screen 104 can include measurements, warnings, and notes for performing the ventilation. In some embodiments, the display screen 104 can include a touch screen that can be used to input information about the patient's size and weight in order to optimize the ventilation for the particular patient. As illustrated in FIG. 7B, The monitoring module can include a display 108 for showing whether rate, volume or pressure are safe, slightly higher or lower than the safe zone, or too high, or too low. This can facilitate quick correction of either rate or pressure being used. The display 108 can use LEDs in colors or that illuminate a colored filter, such as green for safe, yellow for beyond safe, and red for unsafe rate or pressure.

The enclosure cannot be airtight because the gauge sensors need to sample atmospheric pressure in order to compare it to the pressure being measured. The position of the sensors onto the circuit board could also interfere with sampling of atmospheric pressure. Depending on final enclosure design or sensor placement on the circuit board a dual port axial barb gauge sensor may be necessary to sample atmospheric pressures outside of an airtight enclosure or position on the circuit board.

The pressure sensors have an error of less than +/−0.25% each un-calibrated. The Hamilton variable-orifice flowmeter has an error of less than +/−10% un-calibrated. The tidal volume range for lung protective strategy is 6-8 cc/kg, and the device uses 7 cc/kg as its target tidal volume to prevent hypoventilation below 6 cc/kg or barotrauma/volutrauma above 8 cc/kg, which would be larger than a +/−10.5% error from 7 cc/kg.

The technology of the present invention has broader applications as a platform. The device of the invention can employ real-time analysis of respiratory parameters such as: lung compliance, pressure/volume loops expanding the devices role to identify and treat lung diseases earlier both pre-hospital and intra-hospital. Similarly, real-time analysis during CPR allows for the treatment of reversible causes of cardiac arrest. Likewise, real-time physician alerts would allow those with less training to identify and treat underlying lung disease sooner.

In some embodiments the device can have a standalone disposable design that includes a built-in power unit, simple-to-read LED red to green indicator display for pressure/rate/volume, as illustrated in FIGS. 7A and 7B, and wireless data communication allowing for wireless transmission to the updated training display. Also, data processing electronics with low power wireless (Bluetooth 4.1) communication protocol to a data management hub. This would allow for data to be sent from the battlefield, disaster site, or a remote area, to a command center or hospital.

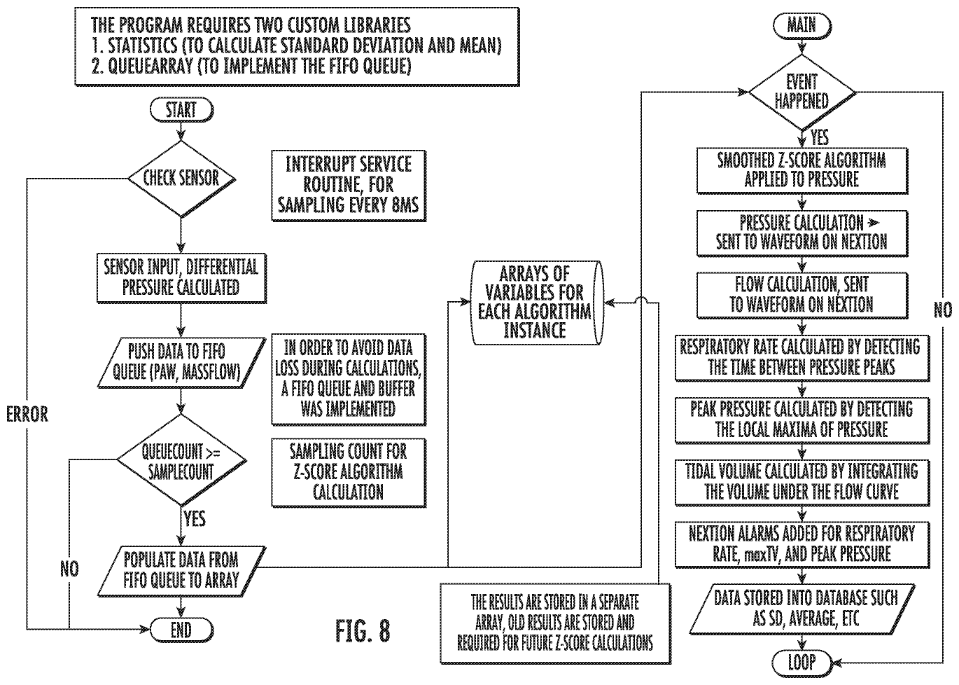

FIG. 8 illustrates a flow diagram of a computer program for operating a device for preventing volutrauma and barotrauma, according to an embodiment of the present invention. The program can be fixed on a non-transitory computer readable medium. The non-transitory computer readable medium can be disposed on a computing device directly connected to a device of the present invention or it can reside on a serve to be accessed remotely, either via a wired or wireless connection. The program is configured to determine respiratory rate and pressure and to alert the care provider to conditions that exceed presets for rate and pressure.

A non-transitory computer readable medium is understood to mean any article of manufacture that can be read by a computer. Such non-transitory computer readable media includes, but is not limited to, magnetic media, such as a floppy disk, flexible disk, hard disk, reel-to-reel tape, cartridge tape, cassette tape or cards, optical media such as CD-ROM, writable compact disc, magneto-optical media in disc, tape or card form, and paper media, such as punched cards and paper tape. The computing device can be a special computer designed specifically for this purpose. The computing device can be unique to the present invention and designed specifically to carry out the method of the present invention. The computing device can also take the form of an operating console computer. The operating console is a non-generic computer specifically designed by the manufacturer. It is not a standard business or personal computer that can be purchased at a local store. Additionally, the console computer can carry out communications with the scanner through the execution of proprietary custom built software that is designed and written by the manufacturer for the computer hardware to specifically operate the hardware.

The many features and advantages of the invention are apparent from the detailed specification, and thus, it is intended by the appended claims to cover all such features and advantages of the invention which fall within the true spirit and scope of the invention. Further, since numerous modifications and variations will readily occur to those skilled in the art, it is not desired to limit the invention to the exact construction and operation illustrated and described, and accordingly, all suitable modifications and equivalents may be resorted to, falling within the scope of the invention.

1. A monitor for a bag valve mask comprising:
an electronic spirometer; and
a display screen.

2. The monitor of claim 1 wherein the electronic spirometer is one selected from a group consisting of an inline spirometer and an out of line spirometer.

3. The monitor of claim 1 further comprising the display screen having a touch screen.

4. The monitor of claim 1 further comprising the electronic spirometer comprising a bi-directional digital turbine.

5. The monitor of claim 4 wherein the bi-directional digital turbine comprises volume, pressure, and respiratory rate alarms.

6. The monitor of claim 4 wherein the bi-directional digital turbine is configured to prevent volutrauma/barotrauma and hypoxia.

7. The monitor of claim 2 wherein the out of line spirometer comprises variable orifice or fixed orifice flowmeter (pitot tube).

8. The monitor of claim 1 wherein the electronic spirometer further comprises a display to indicate whether respiratory rate and pressure are within a predetermined safe zone.

9. The monitor of claim 1 further comprising a non-transitory computer readable medium programmed for determining respiratory rate and pressure.

10. The monitor of claim 9 wherein the non-transitory computer readable medium is configured to communicate wirelessly with the electronic spirometer.

11. The device of claim 1 wherein the electronic spirometer is configured for use with one chosen from a group consisting of a bag valve mask (BVM), endotracheal tube, a Mapleson circuit, an airway management device.

12. A device for delivering positive pressure ventilation comprising:
a mask;
a self-inflating bag;
an electronic spirometer; and
a display screen.

13. The device of claim 12 wherein the electronic spirometer is one selected from a group consisting of an inline spirometer and an out of line spirometer.

14. The device of claim 12 further comprising the display screen having a touch screen.

15. The device of claim 12 further comprising the electronic spirometer comprising a bi-directional digital turbine.

16. The device of claim 15 wherein the bi-directional digital turbine comprises volume, pressure, and respiratory rate alarms.

17. The device of claim 15 wherein the bi-directional digital turbine is configured to prevent volutrauma/barotrauma and hypoxia.

18. The device of claim 13 wherein the out of line spirometer comprises pitot tubes.

19. The device of claim 12 further comprising a non-transitory computer readable medium programmed for determining respiratory rate and pressure.

20. The device of claim 19 wherein the non-transitory computer readable medium is configured to communicate wirelessly with the electronic spirometer.
